(19) United States
(12) Patent Application Publication
(10) Pub. No.: US 2016/0089203 A1
(43) Pub. Date: Mar. 31, 2016

(54) IRRADIATION DEVICE

(71) Applicant: TERUMO KABUSHIKI KAISHA, Tokyo (JP)

(72) Inventors: Katsuhiko SHIMIZU, Fujinomiya-city (JP); Yuuichi TADA, Tokyo (JP); Yuuki ITOU, Hadano-city (JP); Kazuyuki TAKAHASHI, Ashigarakami-gun (JP)

(73) Assignee: TERUMO KABUSHIKI KAISHA, Tokyo (JP)

(21) Appl. No.: 14/869,442

(22) Filed: Sep. 29, 2015

(30) Foreign Application Priority Data
Sep. 29, 2014 (JP) 2014-198764

(51) Int. Cl. A61B 18/22

(52) U.S. Cl. CPC A61B 18/22; A61B 2018/00404

(57) ABSTRACT

There is provided an irradiation device which irradiates light on an inner side face of a biological lumen. The irradiation device includes an optical fiber through which light passes, and a plurality of optical members each having a spherical shape. The plurality of optical members are arrayed in a line along a direction in which light emitted from a distal end of the optical fiber is radiated so that the light is radiated toward the inner surface of the biological lumen.

### CROSS-REFERENCES TO RELATED APPLICATIONS

This application claims priority to Japanese Application No. 2014-198764 filed on Sep. 29, 2014, the entire content of which is incorporated herein by reference.

### TECHNICAL FIELD

The present disclosure generally relates to an irradiation device which irradiates light upon an inner side face of a tube.

### BACKGROUND DISCUSSION

As one of methods for treating a varicose vein, a laser ablation method is available. According to the laser ablation method, an optical fiber is inserted into a blood vessel and laser light emitted from the optical fiber is irradiated upon an inner side face of the blood vessel to cauterize the inner side face of the blood vessel thereby to occlude the blood vessel. Japanese Patent Laid-Open No. 2008-224979 proposes an optical fiber for use with the laser ablation method.

### SUMMARY

In the laser ablation method, in order to prevent laser light from being concentrated upon part of an inner side face of a blood vessel and damaging the blood vessel, the laser light is preferably irradiated uniformly and over a wide range on the inner side face of the blood vessel.

The irradiation device disclosed here is advantageous with respect to the irradiation of light upon the inner surface of a blood vessel.

An irradiation device which irradiates light on an inner surface of a tube includes: an optical fiber through which light passes, and a plurality of optical members positioned distally of the distal end of the optical fiber. Each of the optical members possesses a spherical shape, and the plurality of optical members is arrayed in a line along a direction in which light emitted from the distal end of the optical fiber is radiated so that the light is radiated toward the inner surface of the tube.

With the present disclosure, a technology which is advantageous in irradiation of light upon an inner surface of a tube (blood vessel) is provided.

Another aspect of the disclosure involves an irradiation device which irradiates light on the inner surface of a blood vessel in a living body. The irradiation device possesses a distal end and comprises an optical fiber through which light is emitted, and a plurality of optical members held in place distally of the distal end of the optical fiber. Each of the optical members possesses an outer diameter smaller than the inner diameter of the optical fiber. The plurality of optical members is positioned axially adjacent one another so that the central axis of the optical fiber passes through each of the plurality of optical members, and each of the plurality of optical members is configured so that light emitted from the distal end of the optical fiber is radiated outwardly toward the inner surface of the blood vessel in the living body.

Another aspect of the disclosure involves a method comprising inserting an irradiation device into a blood vessel in a living body, wherein the irradiation device comprises an optical fiber and a plurality of optical members, with the optical fiber possessing a distal end and the optical embers being arranged distal of the distal end of the optical fiber. The method further involves emitting light from the distal end of the optical fiber toward the plurality of optical members so that the light enters each of the optical members and is radiated outwardly toward the inner surface of the blood vessel in the living body.

### BRIEF DESCRIPTION OF THE DRAWINGS

FIG. 1 is a perspective view depicting an example of a configuration of an irradiation device according to a first embodiment.

FIG. 2 is a longitudinal cross-sectional view depicting an example of the configuration of the irradiation device depicted in FIG. 1.

FIG. 3 is a longitudinal cross-sectional view depicting an example of the configuration of the irradiation device depicted in FIG. 1.

FIG. 4 is a longitudinal cross-sectional view depicting an example of the configuration of the irradiation device depicted in FIG. 1.

FIG. 5 is a longitudinal cross-sectional view depicting an example of the configuration of the irradiation device depicted in FIG. 1.

FIG. 6 is a longitudinal cross-sectional view depicting an example of the configuration of the irradiation device depicted in FIG. 1.

FIG. 7 is a longitudinal cross-sectional view depicting an example of the configuration of the irradiation device depicted in FIG. 1.

FIG. 8 is a longitudinal cross-sectional view depicting an example of a configuration of an irradiation device according to a second embodiment

FIG. 9 is a longitudinal cross-sectional view depicting an example of a configuration of an irradiation device according to a third embodiment.

### DETAILED DESCRIPTION

In the following, preferred embodiments of the irradiation device representing examples of the inventive irradiation device disclosed here are described with reference to the accompanying drawings. It is to be noted that, in the figures, like members or elements are denoted by like reference symbols and a detailed description of features already described is not repeated. Further, while the following description of the embodiments is directed to an irradiation device which is inserted into the inside of a blood vessel and irradiates light upon the inner side face of the blood vessel, the tube into which the irradiation device is to be inserted is not limited to a blood vessel.

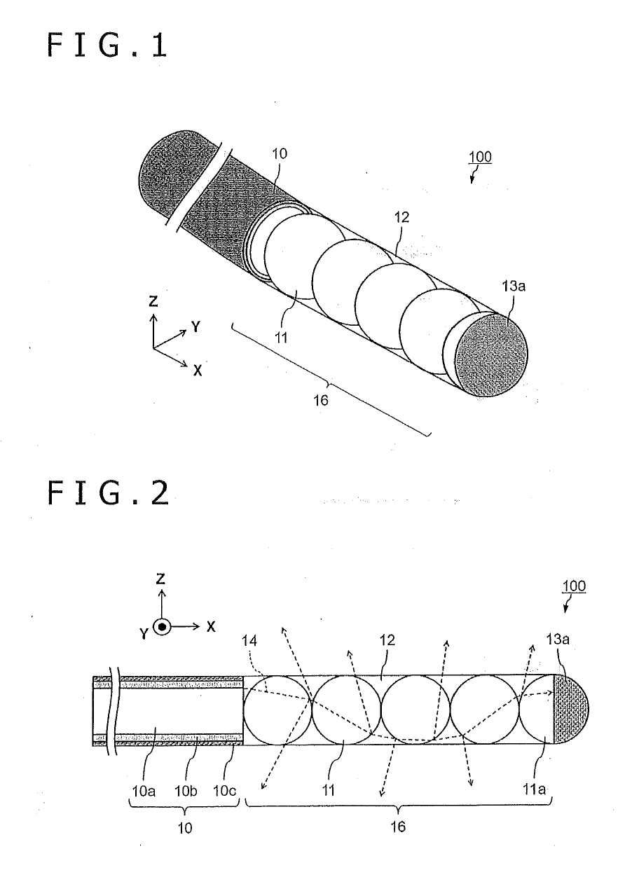

An irradiation device 100 according to a first embodiment of the present disclosure is described. FIG. 1 is a schematic perspective view depicting an example of a configuration of the irradiation device 100 of the first embodiment. FIG. 2 is a longitudinal cross-sectional view depicting the example of the configuration of the irradiation device 100. The irradiation device 100 can be used such that it is inserted into the inside of a blood vessel (biological lumen) and irradiates light (laser light) upon an inner side face (inner periphery or inner surface of the blood vessel/biological lumen) of the blood vessel in a laser ablation method which is one of methods for treating a varicose vein. By using the irradiation device 100 to cauterize the inner side face of the blood vessel, the blood vessel can be occluded to treat a varicose vein.

Referring to FIGS. 1 and 2, the irradiation device 100 can include, for example, an optical fiber 10 through which light (laser light) passes and a plurality of optical members 11. In the illustrated embodiment, the optical members 11 are positioned distally of the distal end of the optical fiber 10. The optical fiber 10 includes, for example, a core 10a along which laser light propagates, a clad 10b which covers or surrounds the core 10a, and a coating membrane 10c which covers or surrounds the clad 10b. The refractive index of the core 10a is higher than (greater than) the refractive index of the clad 10b to allow laser light to propagate in the inside of the core 10a. The core 10a and the clad 10b of the optical fiber 10 can be configured, for example, from quartz glass or plastics.

The optical members 11 are arrayed or positioned in a line along a direction (for example, in an X direction) in which laser light 14 emitted from the distal end of the optical fiber 10 can be radiated so that the laser light may be radiated toward an inner side face of a blood vessel. In particular, the optical members 11 are arrayed such that the centers of the optical members 11 are disposed on an extension line of the center axis of the optical fiber 10. That is, the center axis of the optical fiber passes through the center of each of the optical members 11. The optical members 11 have an outer profile of an independent three-dimensional shape. In the illustrated embodiment, each of the optical members 11 has a spherical shape possessing an outer diameter smaller than the inner diameter of the cross section of the optical fiber 10 and may be configured from quartz glass, plastic or an air layer. Each of the optical members 11 may have a hemispherical shape, a triangular pyramid shape or the like. By setting the diameter of each of the optical members 11 to a dimension smaller than the inner diameter of the cross section of the optical fiber 10, it is possible to smoothly move the irradiation device 100 on the inner side of a blood vessel. Preferably, the optical members 11 are configured such that the refractive indexes of the optical members 11 are higher than that of the core 10a of the optical fiber 10. By configuring the optical members 11 in this manner, it is possible to reflect the laser light 14 emitted from the distal end of the optical fiber 10 by the surface or the inside of the optical members 11 to radiate the laser light 14 toward the inner side face of the blood vessel.

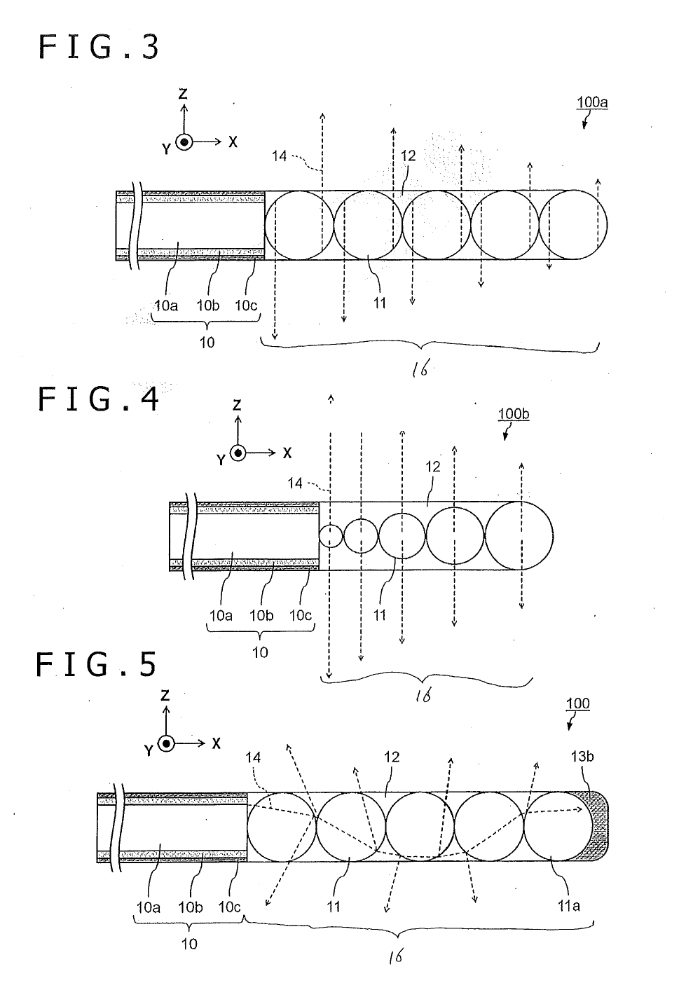

Here, the optical members 11 may be configured such that they possess or exhibit refractive indexes which gradually increase from the distal end side toward the proximal end side (optical fiber side) of an irradiation device 100a as depicted in FIG. 3. In FIG. 3, the distal end side or distal end is the right end, and the proximal end side or proximal end is the left end. Also in FIG. 3, the laser light directed outwardly by the optical members 11 is indicated by the dotted lines, and the illustrated length of the dotted lines indicates the amount of intensity of the laser light, with longer length dotted lines indicating greater intensity of laser light. The illustrated dotted lines in the other drawing figures similarly depict the laser light directed outwardly by the optical members 11 and the intensity of laser light. FIG. 3 depicts the irradiation device 100a in which the refractive index of each of the optical members 11 gradually increases from the distal end side toward the proximal end side. Thus, the optical members 11 toward the left end of the irradiation device 100a in FIG. 3 exhibit a greater or larger refractive index compared to the optical members 11 toward the right end of the irradiation device 100a in FIG. 3. If the optical members 11 are configured in this manner, then the intensity of the laser light 14 emitted from the irradiation device 100a can be gradually increased from the distal end side toward the proximal end side. Further, if the laser light 14 is irradiated upon an inner side face of a blood vessel while the irradiation device 100a is moved in a longitudinal direction (axial direction) of the irradiation device 100a along the inner side face of the blood vessel, then it is possible to quickly raise the temperature of the inner side face of the blood vessel to that within a temperature range suitable for degeneration of the organization of the inner side face of the blood vessel and keep the temperature range suitable for the degeneration (without deviation from the temperature range) for a fixed period of time. Therefore, it is possible to cauterize and degenerate the inner side face of the blood vessel suitably. Alternatively, the plurality of optical members 11 may be configured such that they have different outer diameters which gradually decrease from the distal end side toward the proximal end side of an irradiation device 100b as depicted in FIG. 4. Since the intensity of the laser light 14 in the optical fiber 10 gradually increases toward a central portion of the optical fiber 10, by configuring the optical members 11 such that the optical members 11 nearer to the optical fiber 10 possesses a smaller outer diameter, the laser light 14 emitted from a central portion of the optical fiber 10 can be emitted toward the inner side face of the blood vessel more efficiently.

A fixing member 12 may be provided in gaps between the axially adjacent optical members 11 and in a gap between the optical fiber 10 and the optical member 11 closest to the optical fiber 10 to fill up the gaps to fix the optical members 11 and the optical fiber 10. The fixing member 12 is, for example, a bonding agent and preferably has a refractive index substantially equal to that of the core 10a of the optical fiber 10. For example, the difference between the refractive index of the fixing member 12 and the refractive index of the core 10a of the optical fiber 10 preferably is within a range of 10% with respect to the refractive index of the core 10a of the optical fiber 10. Further, the fixing member 12 preferably is configured so as to have elasticity. If the fixing member 12 is configured so as to have elasticity in this manner, then a distal end portion of the irradiation device 100 (which is a portion which includes the plurality of optical members 11 and at which the optical members 11 are disposed independently of each other) can be curved or bent in accordance with the shape of the blood vessel.

Here, if the laser light 14 is emitted from the optical fiber 10 and passes through the plurality of optical members 11 and thereupon is emitted in the X direction from an optical member 11a which is positioned farthest among the optical members 11 from the optical fiber 10, then the laser light 14 can be irradiated upon a place different from a place upon which the laser light 14 is to be irradiated on the inner side face of the blood vessel. As a result, it becomes difficult to control the irradiation amount of the laser light 14 upon the different place, and the blood vessel may be damaged by the laser light. Therefore, the irradiation device 100 may include a reflecting member which reflects light emitted from the optical fiber 10, passing through the optical members 11 and emitted in the X direction from the optical member 11a which is positioned farthest from the optical fiber 10. The reflecting member may include a reflecting film 13a (for example, a metal film) provided on a face from within the surface of the optical member 11a, which is positioned farthest from the optical fiber 10, on the opposite side to the optical fiber 10 as depicted in FIGS. 1 and 2. Alternatively, the reflecting member may include a mirror 13b disposed farther than the optical member 11a positioned farthest from the optical fiber 10 as depicted in FIG. 5. Where the reflecting member is provided in this manner, the laser light 14 is suppressed from being emitted in the X direction from the optical member 11a, and irradiation of the laser light 14 upon the inner side face of the blood vessel can be controlled with a relatively high degree of accuracy. In addition, it is possible to allow the laser light 14 reflected from the reflecting member to pass through the optical members 11 again and to be irradiated upon the inner side face of the blood vessel.

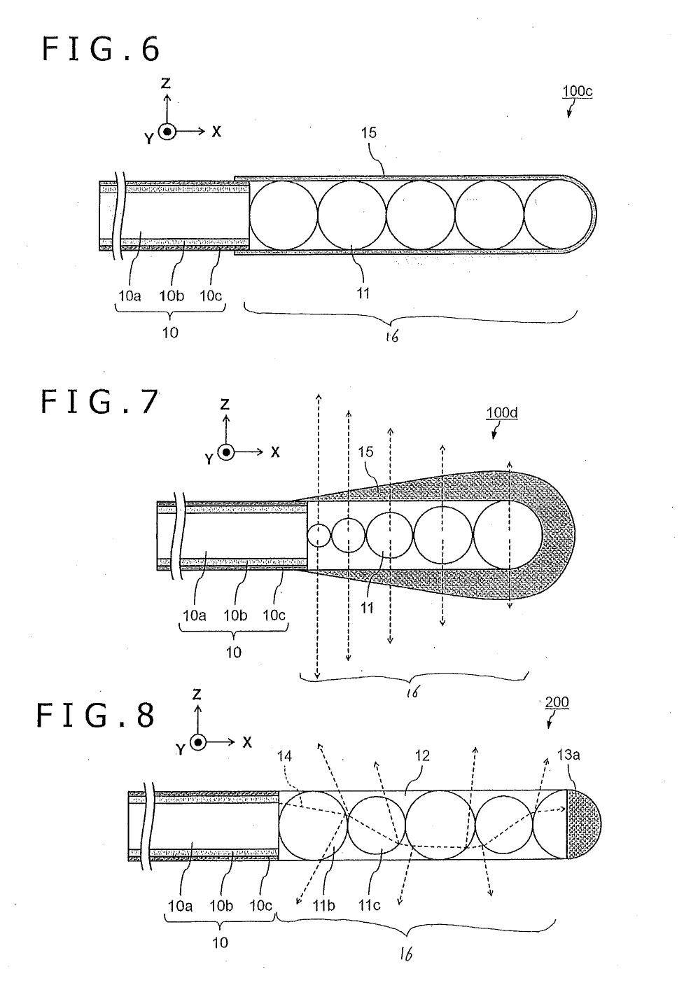

The irradiation device 100 may otherwise have a cap 15 which covers the optical members 11 as depicted in FIG. 6 and through which the laser light 14 passes. FIG. 6 depicts an irradiation device 100c on which the cap 15 is provided. By virtue of the cap 15, the optical members 11 are held in position and can be prevented from being broken. Further, the irradiation device 100 may be configured such that the thickness of the cap 15 decreases from the distal end side toward the proximal end side as depicted in FIG. 7. FIG. 7 depicts an irradiation device 100d provided with a cap 15 whose thickness decreases from the distal end side toward the proximal end side. The cap 15 thus possesses a varying thickness so that the thickness of the cap 15 gradually decreases from the most distal one of the optical members 11 towards the optical fiber 10. At this time, the cap 15 may be configured from a substance by which the laser light 14 is absorbed or may have a portion configured from a substance (for example, glass, carbon or the like) by which the laser light 14 is absorbed. Where the cap 15 having such a configuration as just described is provided, the intensity of the laser light 14 emitted from the irradiation device 100 can be gradually increased from the distal end side toward the proximal end side. Further, where the thickness of the cap 15 is fixed from the distal end side toward the proximal end side (i.e., where the thickness of the cap 15 is constant from the distal end side to the proximal end side), if the content of the substance by which the laser light 14 is absorbed gradually increases toward the distal end side over the range from the distal end side to the proximal end side of the cap 15, then the intensity of the laser light 14 emitted from the irradiation device 100 is gradually increased from the distal end side toward the proximal end side. Then, if the laser light 14 is irradiated upon the inner side face of the blood vessel while the irradiation device 100a is moved in the longitudinal direction (axial direction) along the inner side face of the blood vessel, then it is possible to quickly raise the temperature of the inner side face of the blood vessel to that within a temperature range suitable for degeneration of the organization of the inner side face of the blood vessel and keep the temperature range suitable for the degeneration (without deviation from the temperature range) for a fixed period of time. Therefore, it is possible to suitably cauterize and degenerate the inner side face of the blood vessel. It is to be noted that, in FIG. 7, the optical members 11 are configured such that the diameter of the optical members 11 gradually decreases from the distal end side toward the proximal end side of the irradiation device 100d. By employing an irradiation device including both optical members 11 with outer diameters that gradually decrease from the distal end side toward the proximal end side and the cap 15 possessing a thickness that gradually reduces from the distal end side toward the proximal end side in this manner, the intensity difference between the laser light 14 emitted from the distal end side of the irradiation device 100d and the laser light 14 emitted from the proximal end side of the irradiation device 100d can be increased.

As described above, the irradiation device 100 of the first embodiment includes the optical fiber 10 through which laser light passes, and the plurality of optical members 11 for radiating the laser light emitted from the optical fiber 10 upon the inner side face of the blood vessel. Consequently, the irradiation device 100 can irradiate the laser light uniformly and over a wide range upon the inner side face of the blood vessel. While the irradiation device 100 in the first embodiment here is directed to an example which includes five optical members 11, the irradiation device 100 is not limited to this. The number of optical members 11 can be suitably determined in accordance with a range within which laser light is to be irradiated. Further, the refractive index of the optical members 11 can be suitably determined in response to the refractive index of the core 10a of the optical fiber 10 and the number of optical members 11.

An irradiation device 200 of a second embodiment is described with reference to FIG. 8. FIG. 8 depicts an example of a configuration of the irradiation device 200 of the second embodiment. In the irradiation device 200 of the second embodiment, a plurality of optical members 11 include a plurality of first optical members 11b and a plurality of second optical members 11c smaller in outer diameter than the first optical members 11b. The plurality of first optical members 11b and the plurality of second optical members 11c are disposed alternately. By disposing the plurality of first optical members 11b and the plurality of second optical members 11c whose sizes are different from each other in this manner, the irradiation device 200 can irradiate laser light upon an inner side face of a blood vessel uniformly with a higher efficiency in comparison with the irradiation device 100 of the first embodiment. Here, the outer diameter of the first optical members 11b may be smaller than that of the cross-section (inner diameter) of the optical fiber 10. By setting the outer diameter of the first optical members 11b smaller than that of the cross-section of the optical fiber 10 in this manner, the irradiation device 200 can be moved smoothly on the inner side of the blood vessel.

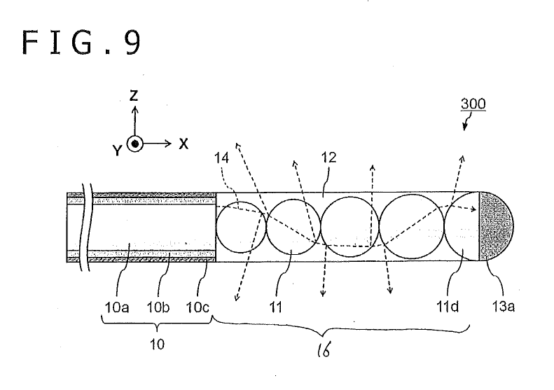

An irradiation device 300 of a third embodiment is described with reference to FIG. 9. FIG. 9 depicts an example of a configuration of the irradiation device 300 of the third embodiment. In the irradiation device 300 of the third embodiment, the outer diameter of a plurality of optical members 11 is set such that it increases away from an optical fiber 10. By applying the configuration just described, laser light can be irradiated upon an inner side face of a blood vessel uniformly with a higher efficiency in comparison with the irradiation device 100 of the first embodiment. Here, the outer diameter of the optical member 11d possessing the greatest or largest outer diameter of the plurality of optical members 11 may be smaller than that of the cross-section (inner diameter) of the optical fiber 10. By setting the outer diameter of the largest optical member 11d smaller than that of the cross-section (inner diameter) of the optical fiber 10 in this manner, the irradiation device 300 can be moved smoothly on the inner side of the blood vessel.

An irradiation device of a fourth embodiment is described. The irradiation device can be configured such that a plurality of first optical members 11 are connected to an optical fiber 10 in an offset relationship from the center axis of the optical fiber 10 so that an emitting portion 16 from which a laser light 14 is emitted may have a curved shape (for example, a J shape). By this configuration, the emitting portion 16 and a vessel wall contact each other. Therefore, the laser light 14 can be prevented from being absorbed by the blood.

The detailed description above describes embodiments of an irradiation device representing examples of the inventive irradiation device disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An irradiation device which irradiates light on an inner surface of a blood vessel in a living body, the irradiation device possessing a distal end and comprising:
an optical fiber through which light is emitted, the optical fiber possessing an inner diameter, a distal end and a central axis;
a plurality of optical members held distally of the distal end of the optical fiber, each of the optical members possessing an outer diameter smaller than the inner diameter of the optical fiber; and
the plurality of optical members being positioned axially adjacent one another so that the central axis of the optical fiber passes through each of the plurality of optical members, each of the plurality of optical members being configured so that light emitted from the distal end of the optical fiber is radiated outwardly toward the inner surface of the blood vessel in the living body.

2. The irradiation device according to claim 1, wherein each of the plurality of optical members possesses a different refractive index, the plurality of optical members being arranged so that the refractive index of each successive optical member gradually increases from the distal end of the irradiation device toward the optical fiber.

3. The irradiation device according to claim 1, wherein the plurality of optical members is held by a fixing member provided in gaps between axially adjacent optical members.

4. The irradiation device according to claim 1, wherein each of the plurality of optical members possesses a different outer diameter, the optical members being arranged so that the outer diameter of each successive optical member gradually decreases from the distal end of the irradiation device toward the optical fiber.

5. The irradiation device according to claim 1, wherein each of the plurality of optical members possesses a center, the central axis of the optical fiber passing though the center of each of the optical members.

6. The irradiation device according to claim 1, wherein the optical members are held by a cap covering the plurality of optical members, the cap possessing a varying thickness so that the thickness of the cap gradually decreases from a most distal one of the optical members towards the optical fiber.

7. The irradiation device according to claim 1, wherein the optical members are held by a cap covering all of the plurality of optical members, the cap possessing a constant thickness along its entire axial extent, the cap comprising a light absorbing substance that absorbs light emitted from the optical fiber, an amount of the light absorbing substance being greater at the distal end of the irradiation device than at a position closer to the distal end of the optical fiber.

8. An irradiation device which irradiates light on an inner surface of a biological lumen, the irradiation device possessing a distal end and comprising:
an optical fiber through which light passes, the optical fiber possessing a distal end;
a plurality of optical members positioned distally of the distal end of the optical fiber, each of the optical members possessing a spherical shape; and
the plurality of optical members being arrayed in a line along a direction in which light emitted from the distal end of the optical fiber is radiated so that the light is radiated toward the inner surface of the biological lumen.

9. The irradiation device according to claim 8, wherein the plurality of optical members each possess a different refractive index, the optical members being arranged so that the refractive index of each successive optical member gradually increases from the distal end of the irradiation device toward the optical fiber.

10. The irradiation device according to claim 9 wherein the plurality of optical members each possess a different outer diameter, the optical members being arranged so that the outer diameter of each successive optical member gradually decreases from the distal end of the irradiation device toward the optical fiber.

11. The irradiation device according to claim 8, wherein the optical fiber possesses a central axis and each of the optical members possesses a center, the central axis of the optical fiber passing though the center of each of the optical members.

12. The irradiation device according to claim 8, wherein the optical fiber includes a core possessing a refractive index, the plurality of optical members possessing respective refractive indexes which are higher than the refractive index of the core the optical fiber.

13. The irradiation device according to claim 8, wherein the plurality of optical members includes one optical member located axially farthest from the optical fiber, and further comprising a reflecting member which reflects light emitted from the optical fiber, passing through the plurality of optical members and then emitted from the one optical member positioned axially farthest from the optical fiber.

14. The irradiation device according to claim 13, wherein the reflecting member includes a reflecting film provided on a surface of the one optical member positioned axially farthest from the optical fiber.

15. The irradiation device according to claim 8, further comprising a cap covering the optical members, the cap possessing a varying thickness so that the thickness of the cap gradually decreases from a most distal one of the optical members towards the optical fiber.

16. The irradiation device according to claim 15, wherein the plurality of optical members each possess a different outer diameter, the optical members being arranged so that the outer diameter of each successive optical member gradually decreases from the distal end of the irradiation device toward the optical fiber.

17. The irradiation device according to claim 8, further comprising a cap covering all of the optical members, the cap possessing a constant thickness along its entire axial extent, the cap comprising a light absorbing substance that absorbs light emitted from the optical fiber, an amount of the light absorbing substance being greater at the distal end of the irradiation device than at a position closer to the distal end of the optical fiber.

18. The irradiation device according to claim 8, wherein each of the optical members possesses a different outer diameter, the optical members being arranged so that the outer diameter of each successive optical member gradually decreases from the distal end of the irradiation device toward the optical fiber.

19. A method comprising:
inserting an irradiation device into a blood vessel in a living body, the irradiation device comprising an optical fiber and a plurality of optical members, the optical fiber possessing a distal end and the optical embers being arranged distal of the distal end of the optical fiber; and
emitting light from the distal end of the optical fiber toward the plurality of optical members so that the light enters each of the optical members and is radiated outwardly toward the inner surface of the blood vessel in the living body.

20. The method according to claim 19, wherein the light radiated outwardly from each of the optical members toward the inner surface of the blood vessel in the living body possesses a different intensity.
